(19) United States
(12) Patent Application Publication
(10) Pub. No.: US 2022/0007982 A1
(43) Pub. Date: Jan. 13, 2022

(54) RING ELECTRODE WITH LOW-MELTING INTERNAL STRUCTURE

(71) Applicant: Heraeus Deutschland GmbH & Co. KG, Hanau (DE)

(72) Inventors: Markus JUNG, Hanau (DE); Oliver KEITEL, Hanau (DE)

(73) Assignee: Heraeus Deutschland GmbH & Co. KG, Hanau (DE)

(21) Appl. No.: 17/374,366

(22) Filed: Jul. 13, 2021

(30) Foreign Application Priority Data
Jul. 13, 2020 (DE) 10 2020 118 373.9

(51) Int. Cl. A61B 5/263; A61N 1/05

(52) U.S. Cl. CPC A61B 5/263; A61B 2562/125; A61N 1/056; A61N 1/0551

(57) ABSTRACT

One aspect relates to a ring electrode for electrical stimulation and/or sensing on the human body, including an outer element and an inner element which is arranged eccentrically within the outer element and is directly connected thereto, wherein the outer element includes a first material, and the inner element includes a second material, the second material having a lower melting point than the first material, wherein the outer element includes a through-opening, and wherein the inner element includes a contacting opening for connecting to a conductor element.

### CROSS-REFERENCE TO RELATED APPLICATION

This Utility Patent Application claims priority to German Application No. 10 2020 118 373.9 filed on Jul. 13, 2020, which is incorporated herein by reference.

### TECHNICAL FIELD

One aspect relates to a manufacturing method for a ring electrode, to a corresponding ring electrode, to an electrode system including such a ring electrode and to a use of the ring electrode or the electrode system in a cardiac pacemaker and/or for neurostimulation. The ring electrode is generally intended for use as or in an active implantable medical device, but may also be used otherwise. It can be used for signal detection and/or for stimulation.

### BACKGROUND

The typically very small component size of a ring electrode for an active implantable medical device and the even smaller dimensions of its partial features require very expensive and complex manufacturing facilities and manufacturing methods with many individual operations. Due to the small component size and the high stability and reliability requirements of medical products for electrical stimulation or sensing, the electrical and mechanical connection between the ring electrode and a conductor is often a particular challenge.

EP3185248A1 describes a method for electrically contacting a coated line with a particle. To this end, the insulation is partially removed and electrically conductive particles are introduced into a window (via) produced in the process. The particle forms a conductive connection between the conductor and a ring electrode surrounding the conductor. However, creep of the plastic insulation can lead to a loss of contact between the line and the particle.

U.S. Pat. No. 7,364,479B1 describes a contacting method which is comparatively complex. US2016303366 A1 describes contacting which uses an additional connecting piece and is therefore also complex.

US20130338745A1 describes contacting by using micro-slides. This is complex and can lead in practice to instability of the contact due to manufacturing tolerances.

US20130338745A1 describes contacting by using ring electrodes which have a plurality of cavities for the electrical conductors. This method is complex and not very flexible.

In conventional methods, it is often not possible to connect, in particular, small structures of conductors and electrodes with an integral bond, for example a welded joint, without severely damaging the plastic insulation of the conductor. In a conventional welded joint at the end of the electrode, the connection is susceptible to fatigue fracture. Ring electrodes with an internal contacting opening for connection to a conductor have the advantage that the connection between the electrode and the conductor is better protected against external influences. However, with conventional methods it is difficult to stably weld together the conductor and the electrode from outside, since the exact position of the conductor in the internal contacting opening is visible only to a limited degree or not at all from the outside.

For these and other reasons there is a need for the present embodiment.

### SUMMARY

An object of one embodiment is to solve one or more of the above and other problems of the prior art. For example, one embodiment allows for a simple and cost-effective manufacture of ring electrodes having a plurality of openings. Furthermore, one embodiment provides ring electrodes having an internal contacting opening which can be easily and securely welded to a conductor element from the outside without damaging the ring electrode.

These objects are achieved by the methods and devices described herein, particularly those described in the claims.

Embodiments are described below.
1. Ring electrode for electrical stimulation and/or sensing on the human body, comprising an exterior wall and a contact element directly connected thereto, said contact element being arranged eccentrically within the exterior wall, said exterior wall comprising a first material, and the contact element comprising a second material, wherein the second material has a lower melting point than the first material, wherein the exterior wall comprises a through-opening, and wherein the contact element comprises a contacting opening for connection to a conductor element.
2. Ring electrode according to embodiment 1, wherein the second material is selected from the group consisting of Pt, Cu, Pd, Ti, Fe, Au, Mo, Ni, MP35N, 316L, 301, 304, and an active solder.
3. Ring electrode according to any one of the preceding embodiments, wherein the first material is selected from the group consisting of Pt, Ir, Ta, Pd, Ti, Fe, Au, Mo, Nb, W, Ni, Ti, MP35N, 316L, 301 and 304.
4. Ring electrode according to any one of the preceding embodiments, further comprising a diffusion barrier between the first material and the second material.
5. Ring electrode according to any one of the preceding embodiments, wherein the absolute melting point [K] of the first material is at least 1.1 times; 1.2 times; 1.3 times; 1.4 times; 1.5 times or at least 2 times the absolute melting point of the second material.
6. Electrode system comprising a ring electrode according to any one of the preceding embodiments and a conductor element, wherein the conductor element is arranged within the contacting opening and is integrally bonded to the contact element and is preferably alloyed therewith.
7. Method for connecting a ring electrode according to any one of embodiments 1 to 6 to a conductor element, comprising the following steps:
Bringing the ring electrode into contact with the conductor element, the conductor element being arranged at least partially within the contacting opening, Heating the contact element and thereby forming an integral bond between the second material and the conductor element, wherein the formation of an integral bond preferably comprises the formation of an alloy.
8. Method according to embodiment 7, wherein heating of the contact element is effected by heating the outer side of the exterior wall and/or to a temperature that lies between the melting point of the second material and the melting point of the first material.
9. Method according to embodiment 7 or 8, wherein heating of the contact element is effected by means of induction heating or local heat input by a laser beam or resistance welding.
10. Method according to any one of embodiments 7 to 9, further comprising compressing the conductor element within the contacting opening to produce a frictional connection between the conductor element and the contact element.
11. Electrode system producible by a method according to any one of embodiments 7 to 10.
12. Manufacturing method for a ring electrode, comprising the following steps:
Providing an outer element comprising an outer tube made of a first material, providing a first inner element comprising a first inner tube and a first core made of a sacrificial material,
Providing a second inner element comprising a second inner tube made of a second material and a second core made of a sacrificial material, said second material having a lower melting point than the first material,
Forming a composite tube by arranging the first inner element and the second inner element within the outer element, the first inner element and the second inner element being arranged eccentrically to each other,
Drawing the composite tube in a longitudinal direction of the composite tube,
Separating a composite tube disc from the composite tube,
Removing the sacrificial material of the first core, and
Removing the sacrificial material of the second core to obtain a contacting opening in the ring electrode.
14. Precursor for a ring electrode, comprising an outer element and an inner element, which is arranged eccentrically within the outer element and is directly connected thereto, wherein the outer element comprises an outer tube made of a first material, and the inner element comprises an inner tube made of a second material, the second material having a lower melting point than the first material.
15. Precursor for a ring electrode according to embodiment 14, wherein the inner tube surrounds a core made of a sacrificial material and the outer element surrounds a further core made of a sacrificial material.
16. Precursor for a ring electrode according to embodiment 14 or 15, further comprising a further inner element comprising a further inner tube and optionally a further core within the further inner tube.

### BRIEF DESCRIPTION OF THE DRAWINGS

Further features, advantages and possible applications of the present embodiments follow from the following description of the exemplary embodiments and the figures. All features described and/or illustrated form the subject matter of the embodiments per se and in any combination, also independently of their composition in the individual claims or their back-references. In the figures, like reference numerals designate like or similar objects.

FIG. 1 illustrates a top view of a ring electrode.

FIG. 2 illustrates a top view of a ring electrode including a diffusion barrier.

FIG. 3 illustrates the connection of a conductor element to a ring electrode by using a thermal method.

FIG. 4 illustrates a precursor for a ring electrode described herein including a removable core.

FIG. 5 illustrates a ring electrode which can be produced from the precursor illustraten in FIG. 4.

FIGS. 6a-6f illustrate a manufacturing method for a ring electrode.

### DETAILED DESCRIPTION

In the following Detailed Description, reference is made to the accompanying drawings, which form a part hereof, and in which is illustraten by way of illustration specific embodiments in which the embodiment may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present embodiment. The following detailed description, therefore, is not to be taken in a limiting sense, and the scope of the present embodiment is defined by the appended claims.

It is to be understood that the features of the various exemplary embodiments described herein may be combined with each other, unless specifically noted otherwise.

In principle, for the embodiments described herein, the elements of which “have” or “comprise” a particular feature (e.g., a material), a further embodiment is always contemplated in which the element in question consists solely of the feature, i.e. includes no further components. The word “comprise” or “comprising” is used herein synonymously with the word “have” or “having”.

Where an element is referred to in the singular form in an embodiment, this also contemplates an embodiment where a plurality of these elements are present. The use of a term for an element in the plural basically also includes an embodiment in which only a single corresponding element is present.

Unless otherwise indicated or clearly excluded from the context, it is possible in principle and is clearly considered hereby that features of different embodiments may also be present in the other embodiments described herein. It is also contemplated, in principle, that all features described herein in connection with a method are also applicable to the products and devices described herein, and vice versa. It is purely for reasons of brevity that all such contemplated combinations not explicitly listed in all cases. Technical solutions which, as is known, are equivalent to the features described herein are also intended to be covered by the scope of the invention.

### Ring Electrode

One aspect of one embodiment relates to a ring electrode for electrical stimulation and/or sensing on the human body, including an exterior wall and a contact element directly connected thereto, the contact element being arranged eccentrically within the exterior wall, the exterior wall including a first material, and the contact element including a second material, wherein the second material has a lower melting point than the first material, wherein the exterior wall includes a through-opening, and wherein the contact element includes a contacting opening for connection to a conductor element.

The contact element can, for example, be a substantially cylindrical tube. In one embodiment, the contact element has a uniform wall thickness. The wall thickness of the contact element may, for example, be at least 1 pm, at least 5, 10, 20, 50, 100, 200, 300, 400 or 500 pm. In one embodiment, the contact element consists solely of a single material, the second material. In one embodiment, the contact element consists solely of the first material that is also contained in the exterior wall and of the second material. In one embodiment, the contact element is free of lead, tin, bismuth and/or silver. In one embodiment, the contact element consists of two or more tubes that have been drawn to form a composite tube. In one embodiment, the contact element is free of tin solder and comparable solder materials. In one embodiment, the contact element is configured to be welded to a conductor element. In one embodiment, the inner side of the contact element consists entirely of the second material.

The exterior wall of the ring electrode is in one embodiment designed to receive or emit an electrical signal. Advantageously, the exterior wall is formed of an electrically conductive and biocompatible first material, such as platinum or a platinum alloy, such as PtIr10 or PtIr20. The exterior wall includes a through-opening, which is in one embodiment configured to receive a cable, the cable in one embodiment including a plurality of electrical conductors, referred to herein as conductor elements. The individual conductor elements may include an electrical insulation on their outer side.

The ring electrode includes a contact element which is mechanically and electrically conductively connected to the exterior wall. The contact element is in one embodiment tubular and includes a second material which has a lower melting point than the first material.

The melting temperature, also referred to as melting point, of a material can either be obtained from the literature or determined with simple experiments. The melting point can be determined using DSC calorimetry. A suitable device for determination is the DSC 204 F1 Phoenix by Nietzsch, Selb, Germany.

The melting temperature described herein is also referred to herein as melting point and refers to the absolute melting temperature measured in Kelvin.

In some embodiments, the absolute melting point [K] of the first material is at least 1.1 times; 1.2 times; 1.3 times; 1.4 times; 1.5 times or at least 2 times the absolute melting point of the second material.

For example, if the second material has a melting point of 1000 K, in this case, 1.5 times the melting temperature is 1000*1.5=1500 K. Accordingly, in one example, the second material could have a melting point of 1000 K and the first material could have a melting point of 1500 K.

Suitable materials for the first material are in particular metals and alloys.

Biocompatible metals or alloys are particularly preferred. For example, the first material may be selected from the group consisting of Pt, Ir, Ta, Pd, Ti, Fe, Au, Mo, Nb, W, Ni, Ti, MP35N, 316L, 301 and 304.

Suitable materials for the second material are in particular metals and alloys. For example, the second material may be selected from the group consisting of Pt, Cu, Pd, Ti, Fe, Au, Mo, Ni, MP35N, 316L, 301, 304, and an active solder.

Examples of groups of suitable active solders are, for example, silver titanium solders, silver copper titanium solders and silver copper indium titanium solders. In one embodiment, the active solder is AgTi4 or AgCuTi3. Further examples of active solders are 96Au4Ti, 98Au2Ti, 50Ti50Ni, 96.4Au3NilTi, 92.75Cu2Al3Si2.25Ti, 67Ti33Ni, 96Ag4Ti, 70Til5Cu15Ni, 98.4AglIn0.6Ti, 60Ti25Nil5Cu, 92.75Ag5CulAl1.25Ti, 68.8Ag26.7Cu4.5Ti, 63Ag35.25Cul.75Ti, 63Ag34.25Cu1.75TilSn, 60.3Ag23Cul4.7ln2Ti, 59Ag27.25Cul2.51n1.25Ti, 43.6Ag29.1Cu24.3In3Ti, and 96.4Au3Ni0.6Ti.

In one embodiment, the second material has a melting point greater than 250° C., greater than 300° C., 400° C., 500° C., 600° C., 700° C., 800° C., 900° C., or greater than 1000° C. In one embodiment, the second material has a hardness of at least 50 HV. In one embodiment, the second material has a tensile strength of at least 200 MPa. In one embodiment, the second material has a modulus of elasticity of at least 100 GPa. In one embodiment, the second material is integrally bonded to the exterior wall. In one embodiment, the second material is free of lead, tin, bismuth and/or silver. In one embodiment, the second material is free of platinum. In one embodiment, the second material is free of lead, tin, bismuth, platinum and/or silver. In one embodiment, the diffusion medium is not tin solder or a comparable solder material.

In one embodiment, the ring electrode includes a diffusion barrier arranged between the first material and the second material. A diffusion barrier is a layer of a material which completely or partially prevents the diffusion of the second material into the first material and/or the diffusion of the first material into the second material. The diffusion barrier in one embodiment includes a material different from the first material and/or the second material. For example, the first material may be a platinum iridium alloy, the second material may include gold, and the diffusion barrier may include nickel. For example, the material of which the diffusion barrier is formed may have a higher melting point than the second material.

In particular, such a diffusion barrier may be useful to prevent or reduce diffusion of the second material into the first material when the second material is heated to connect to a conductor element, as described in more detail below.

Due to the low melting point of the second material, a conductor element can be connected to the contact element by using a thermal method without impairing the first material of the exterior wall. The thermal method is in one embodiment a welding method, for example laser or resistance welding.

Some possible dimensions of the ring electrode are given below. The individual dimensions are to be understood independently of one another, and while they do not necessarily form a common embodiment, they may do so. An outer diameter of the ring electrode and thus an outer diameter of the outer element and of the outer tube may be between 1 and 3 mm, in one embodiment between 1.3 and 2.5 mm and in one embodiment between 1.5 and 2.0 mm. An inner diameter of the first inner element and thus an inner diameter of the first inner tube may be between 0.9 and 2.9 mm, in one embodiment between 1.2 and 2.4 mm and in one embodiment between 1.4 and 1.9 mm. An inner diameter of the contacting opening and thus an outer diameter of the second core may be between 0.10 and 0.30 mm, in one embodiment between 0.15 and 0.25 mm and in one embodiment between 0.17 and 0.20 mm.

Given that it is possible to selectively melt and/or activate only the second material almost independently of the dimensions of the ring electrode described herein, particularly small ring electrodes can be produced, which have a particularly small wall thickness of the exterior wall, a small inner diameter of the through-opening, and/or a particularly small outer diameter of the exterior wall, for example. In one embodiment, the outer diameter of the outer tube is 0.3 to 3.0 mm, in one embodiment 0.5 to 2 mm. In one embodiment, the inner diameter of the second inner tube is 0.02 to 0.3 mm, in one embodiment 0.04 to 0.2 mm. In one embodiment, the length of the ring electrode is 0.05 to 5 mm, in one embodiment 0.1 to 3 mm. In one embodiment, the wall thickness of the ring electrode is 0.005 to 0.2 mm, in one embodiment 0.01 to 0.1 mm. For example, the inner diameter of the through-opening may be 0.02 to 0.30 mm, in one embodiment 0.04 to 0.20 mm. For example, the outer diameter of the exterior wall may be 0.3 to 3.0 mm, in one embodiment 0.5 to 2.0 mm.

As described hereinafter, such a ring electrode may be produced by a drawing process from a precursor, for example from a composite tube including an outer element and one or more inner elements. The outer element may include an outer tube. The inner elements may each include an inner tube and/or a removable core, as explained in more detail below. In one embodiment, a ring electrode produced from such a precursor has a boundary line or boundary surface, interface, or “seam” between the outer element and the first inner element when viewed in a cross-section. This can be understood to mean that the outer element and the first inner element do not merge into one another and fuse with one another completely homogeneously and continuously, but that the two elements can still be recognized as originally different components under the microscope.

In one embodiment, the second material is directly connected to an electrically conductive material, for example, the first material, on at least two opposing surfaces. In one embodiment, all solid materials directly connected to the second material are electrically conductive. In one embodiment, all solid materials directly connected to the second material are metals or alloys. In one embodiment, the second material connects a plurality of electrically conductive layers to one another. In one embodiment, the second material is not directly connected to a solid electrically insulating material.

A further aspect of one embodiment relates to a microelectrode or microelectrode array including a ring electrode described herein.

The ring electrodes and their precursors described herein do not necessarily have to have a circular cross section. The cross section of the ring electrodes may be oval or elliptical, for example. The outer surface and the inner surface of the ring electrode in the region of the large through-opening do not necessarily have to be parallel. For example, the cross section of the outer surface may be circular, and the cross section of the inner surface may be elliptical. An angular shape of the cross section is also possible in principle.

The same applies to the used outer elements, inner elements and the components thereof.

Furthermore, an electrode system is described which includes a ring electrode described herein and a conductor element, the conductor element being arranged within the contacting opening and integrally bonded to the contact element. In one embodiment, the conductor element is interlockingly connected and integrally bonded to the contact element. In one embodiment, the conductor element is alloyed with the contact element, i.e. the second material has formed an alloy with a material of the conductor element, for example by fusion or a diffusion process. In one embodiment, the conductor element is welded to the contact element. In one embodiment, the conductor element is fused to the contact element. In one embodiment, the contact element is connected to the conductor element by a liquefaction of the contact element and the conductor element. The conductor element may be connected to the contacting opening or the fastening element of the ring electrode by welding, in particular laser welding or resistance welding, soldering, crimping or the like. In this way, a particularly secure and simple fastening of the conductor element to the ring electrode is achieved. In one embodiment, the conductor element is frictionally connected and integrally bonded to the contacting opening, for example by welding and crimping.

It is further proposed to use a ring electrode described herein or an electrode system described herein, which can be produced, for example, according to the manufacturing methods described here, in a stimulator, such as a cardiac pacemaker, or for neurostimulation. One embodiment can be used as a stimulation or measuring electrode for cardiac pacemaker electrodes, in particular for ventricular, atrial and left ventricular leads. One embodiment can also be used for neurostimulation, for example in spinal cord stimulation, gastric stimulation, peripheral nerve stimulation or deep brain stimulation. Furthermore, use on catheters is possible in electrophysiology applications, for example, such as for ablation, cardiac current measurement or the like. Other uses are also possible, of course.

Examples of catheters according to one embodiment are those which are designed for electrophysiological mapping or ablation of tissue. In one embodiment, the ring electrode is configured and/or intended to be connected to a generator of an active implantable device. A ring electrode of one embodiment can also be used in a stimulator, i.e. a medical device for recording an electrical signal. A stimulator is a medical device which can achieve a physiological effect by emitting an electrical signal to the body of a living being. For example, a neurostimulator may cause an electrical signal in the nerve cell (e.g. an action potential) by delivering an electrical signal to a nerve cell.

One embodiment further relates to the use of a low-melting material in a ring electrode which has a higher-melting material in order to connect a conductor element to the ring electrode by using heating.

### Connection of a Conductor Element to the Ring Electrode

One embodiment also relates to a method for connecting a ring electrode described herein to a conductor element, including the following steps:

Bringing the ring electrode into contact with the conductor element, the conductor element being arranged at least partially within the contacting opening, Heating the contact element, thereby forming an integral bond between the second material and the conductor element. The formation of an integral bond in one embodiment includes the formation of an alloy between the second material of the contact element and a material of the conductor element. The contact element is in one embodiment heated by heating the exterior wall. For example, the exterior wall of the ring electrode may be heated in spatial proximity to the contact element by using a laser or resistance welding device. In this case, the heat is emitted from the outer side of the ring electrode to the internal contact element. The exterior wall and the contact element in one embodiment each include materials with high thermal conductivity.

By heating the second material to a temperature below the melting point of the first material, in one embodiment to a temperature above the melting point of the second material, a conductor element can be connected to the ring electrode 10 within the contacting opening.

In one embodiment, the contact element is heated to a temperature which is between the melting point of the second material and the melting point of the first material.

The conductor element can be connected to the contacting opening by using a thermal method. Examples of a thermal method are welding methods such as laser welding or resistance welding. A further thermal method is diffusion bonding. Diffusion bonding is generally understood to mean a process in which two bodies of different materials which are otherwise difficult to connect to one another are brought into a stable connection. Here, two different materials are brought into contact under suitable temperature and pressure conditions and are held under these conditions for a certain period of time. Under these temperatures and pressures, which are usually elevated compared to normal conditions, on the connecting surface of the two materials, a mass transfer takes place between the two bodies, which can produce a very stable connection between the two bodies. Such a connection is also included herein in the term “integral bond”.

When bonded by diffusion bonding, the second material and the conductor element may be heated, for example, to at least 50%, in one embodiment at least 60% or at least 65% of the melting temperature of the second material. The composite tube is heated, for example, to 50 to 80%, 60 to 70% or 65 to 70% of the melting temperature of the second material.

The temperature may also be selected as a function of the selection of the materials of the conductor element and the ring electrode, in particular of the contact element. In one embodiment, a temperature corresponding to approximately 50-90% of the material of the conductor element is selected. For the materials according to one embodiment, this temperature may be, for example, between 100° C. and 3000° C., in one embodiment between 500° C. and 2700° C., in one embodiment between 700° C. and 2500° C.

In one embodiment, the connection of the ring electrode to the conductor element includes compressing the conductor element within the contacting opening to produce a frictional connection between the conductor element and the contact element. For example, compressing may be carried out by crimping, swaging (die forging) or by pressing together the contact element with pliers. In one embodiment, the conductor element is first compressed within the contacting opening and then welded within the contacting opening.

### Manufacturing Method

One embodiment further relates to a method for manufacturing a ring electrode, including the following steps:
a) Providing an outer element including an outer tube made of a first material,
b) Providing a first inner element including a first inner tube and a first core made of a sacrificial material,
c) Providing a second inner element including a second inner tube made of a second material and a second core made of a sacrificial material, the second material having a lower melting point than the first material,
d) Forming a composite tube by arranging the first inner element and the second inner element within the outer element, the first inner element and the second inner element being arranged eccentrically to each other,
e) Drawing the composite tube in a longitudinal direction of the composite tube,
f) Separating a composite tube disc from the composite tube,
g) Removing the sacrificial material of the first core, and
h) Removing the sacrificial material of the second core to obtain a contacting opening in the ring electrode.

The steps of the method may be performed in the order indicated above or in a different order.

In one embodiment, a material of the outer element and a material of the first inner element have a similar degree of deformation and/or a microstructure similar to each other and/or a similar hardness. For example, the material of the outer tube and the material of the first inner tube may have a microstructure similar to each other. This means, for example, that in each case the crystal grains of a metal have a similar size and/or shape in both materials.

The degree of deformation or natural strain can be understood as the logarithmic ratio of the length of a sample after deformation to a length of the sample before deformation.

If an inner component, e.g. the first inner tube, has the same or higher Vickers hardness as compared to an adjacent external component, e.g. the outer tube, this can improve the stability of the ring electrode produced. In particular, delamination of the outer tube and the first inner tube can be prevented or reduced.

In some embodiments, the C:D ratio is from 0.8 to 1.0; in one embodiment from 0.9 to 1.0; from 0.95 to 1.0, or from 0.99 to 1.0, wherein C is the hardness of the material of the outer tube and D is the hardness of the material of the inner tube. Vickers hardness can be determined by the test methods described hereinbelow.

In one embodiment, drawing of the composite tube takes place with a degree of deformation of between 3 and 30% per individual draw and in one embodiment with a degree of deformation of between 3 and 20% per individual draw. In the overall composite after several or all draws, the degree of deformation may be between 50 and nearly 100%.

In one embodiment, the outer tube and/or one or all of the inner tubes are soft-annealed prior to drawing to promote flowing of the individual tubes into free spaces between the individual tubes.

The outer tube, the first inner tube and/or optionally the second inner tube may each include a metal, for example a noble metal or a base metal. Examples of preferred metals are Pt, Ir, Ta, Pd, Ti, Au, Mo, Nb, W, Ni, Ti, MP35, 316L, 301, 304, as well as alloys of these metals and multilayer material systems.

In some embodiments, the outer tube, the first inner tube, and/or the second inner tube include one or more of the metals Pt, Ir, Ta, Pd, Ti, Fe, Au, MP35N, or a mixture or alloy thereof. In some embodiments, the outer tube, the first inner tube, and/or the second inner tube include the alloys MP35, PtIr10, PtIr20, 316L, 301 or nitinol. The outer tube, the first inner tube and/or the second inner tube may also include multilayer material systems. In one embodiment, the outer tube, the first inner tube and/or the second inner tube include MP35, Au, Ta, Pt, Ir, Pd or Ti. In some embodiments, the outer tube, the first inner tube, and/or the second inner tube contain less than 3%, 2%, or less than 1% Fe.

MP35 is a nickel-cobalt-based hardenable alloy. A variant of MP35 is described in industry standard ASTM F562-13. In one embodiment, MP35 is an alloy including 33 to 37% Co, 19 to 21% Cr, 9 to 11% Mo and 33 to 37% Ni.

PtIr10 is an alloy of 88 to 92% platinum and 8 to 12% iridium.

PtIr20 is an alloy of 78 to 82% platinum and 18 to 22% iridium.

316L is an acid-resistant CrNiMo austenitic steel with approx. 17% Cr; approx. 12% Ni and 2.0% Mo. A variant of 316L is described in industry standard 10088-2. In one embodiment, 316L is an alloy including 16.5 to 18.5% Cr; 2 to 2.5% Mo and 10 to 13% Ni.

301 is a chromium nickel steel with high corrosion resistance. A variant of 301 is described in industry standard DIN 1.4310. In one embodiment, 301 is an alloy including 16 to 18% Cr and 6 to 8% Ni.

Nitinol is a shape memory nickel titanium alloy having an ordered cubic crystal structure and a nickel content of approximately 55%, the remaining portion consisting of titanium. Nitinol has good biocompatibility and corrosion resistance properties. Unless otherwise indicated, all percentages given herein are to be understood as weight percent (wt. %).

The outer tube, the first inner tube, and/or optionally the second inner tube may each independently include or consist of one or more of the above-mentioned metals and alloys. In one embodiment, the outer tube, the first inner tube, and/or optionally the second inner tube include the same metal or alloy. In this case, however, it is provided that in any case the inner element which later forms a contacting opening additionally includes a material having a lower melting point. In one embodiment, the outer tube, the first inner tube and/or optionally the second inner tube each include different materials, for example different metals or alloys. For example, the outer tube and the first inner tube may each include a noble metal; or the outer tube may include a noble metal while the first inner tube includes a base metal. If, for example, the outer tube and the inner tube include the same material at least on the contact surface of these two elements, a particularly firm connection between them can be achieved. In one embodiment, the first inner tube and/or optionally the second inner tube includes titanium. In one embodiment, the first inner tube includes titanium, and the sacrificial material in the first inner tube includes copper. In one embodiment, the second inner tube includes titanium, and the second sacrificial material includes copper. In one embodiment, the outer tube includes platinum, and the first inner tube and/or optionally the second inner tube includes titanium. In one embodiment, the outer tube includes platinum, and the first inner tube and/or optionally the second inner tube includes a base metal.

An advantage of the method described above is that the ring electrode does not have to be made from the full material, such as, for example, from a rod material, but can be produced directly from hollow tubes. In this way, it is possible to dispense with a machining or ablative processing of the outer diameters of the tubes, and significantly less noble metal is used and lost inside the ring electrode, since the tubes have no noble metal core that has to be cleared. This eliminates not only the machining and the clearing costs and effort, but also the costs of the noble metal and noble metal losses.

The contacting opening in the ring electrode can serve for electrical and/or mechanical contacting with a conductor element. The contacting opening can thus serve as an electrical connecting element and/or as a mechanical fastening element for the conductor element. The conductor element may be a cable or a wire for contacting the ring electrode with a medical device such as a cardiac pacemaker.

The composite tube may be produced by inserting the first inner element and the second inner element into the outer element. Here, a defined boundary surface can be produced with, for example, a defined material quality between the outer element, the first inner element and/or the second inner element. For example, a defined material quality of the boundary surface of the contacting opening for the conductor element can be created, so that the contacting of the conductor element on the ring electrode can be particularly secure and reproducible, for example by crimping, clamping or insertion.

The eccentric arrangement of the first inner element and the second inner element relative to one another can be understood such that the center points or centroids of the two inner elements do not lie one on top of the other in cross section. The first inner element and the second inner element are therefore not arranged concentrically and therefore do not form the shape of a target disk. The one inner element can at least partially cover the other inner element and the two inner elements lie next to one another, but in cross section, they have no common center point or centroid. In this way, the contacting opening can be formed such that it lies outside the center point of the ring electrode when viewed in cross section.

Drawing or drawing through can be understood to be a push-pull forming, in which a starting wire is brought to a reduced diameter in a plurality of steps by a die, drawing die or matrix. When drawing the composite tube, the outer and inner elements may flow toward each other and reduce and possibly even close free spaces between them. For example, the first inner tube may engulf the second inner element such that the second inner element extends nose-like into the first inner tube.

By drawing, it is possible at least in part to achieve an interlocking connection and/or a frictional connection between the individual components of the composite tube, so that an end geometry of the ring electrode is stable according to the present manufacturing method. This can be understood to mean that the individual components of the composite tube hold together by mutual mechanical blocking and/or friction. By drawing, it is also possible, at least in part, to achieve an integral bond, for example by cold-welding the individual components of the composite tube. This can be understood to mean that the individual components of the composite tube hold together by chemical or atomic connection. In one embodiment, the individual components of the composite tube are completely or substantially completely bonded to one another after drawing, so that a uniform material composite is present, wherein the individual layers may only be visible through a boundary surface, as described herein.

In one embodiment, the outer element and the first inner element are arranged concentrically to one another. This can be understood to mean that the center points or centroids of the outer element and of the first inner element lie one on top of the other in cross section. In this way, a cylindrical main opening of the ring electrode can be formed.

In one embodiment, the diameter of the first inner element is larger than the diameter of the second inner element. In one embodiment, the diameter of the first inner element is more than twice the diameter of the inner element. In one embodiment, the diameter of the first inner element is more than three times the diameter of the second inner element. In this way, the through-opening of the ring electrode formed by the first inner element is markedly larger than the contacting opening formed by the second inner element.

In one embodiment, removing the sacrificial material of the first core includes mordanting or etching. In one embodiment, removing the sacrificial material of the sacrificial material of the second core includes mordanting or etching. Removal of the sacrificial material of the first core and removal of the sacrificial material of the second core may be performed by the same or different type of mordanting or etching. Mordanting can be understood to mean treating the ring electrode or its components by using a mordant. Aggressive chemicals such as acids or alkalis can be used as mordant. Etching may be understood to mean removing material of the ring electrode or its components by the use of an etchant. Chemical substances which change (usually oxidize) the material to be etched in a chemical reaction and thus bring it into solution can be used as etchants. Etchants may be acids or strong oxidants. Mordanting or etching may be assisted by ultrasound, heat and/or electrical current.

In one embodiment, the sacrificial material of the first core is removed using an acid. In one embodiment, the sacrificial material of the second core is removed using an acid. In both cases, it is possible, but not mandatory, to use the same acid. The acid may be nitric acid, hydrochloric acid, hydrogen peroxide and/or the like.

In one embodiment, the second inner element includes a second inner tube that includes the second core. When drawing the composite tube, the second inner tube can flow into free spaces between the outer tube and the first inner tube. The second inner tube and/or the first inner tube may be soft-annealed to promote such flowing.

In one embodiment, the outer tube includes a noble metal or a noble metal alloy. In one embodiment, the first inner tube includes a noble metal or a noble metal alloy. In one embodiment, the optional second inner tube includes a noble metal or a noble metal alloy. The outer tube, the first inner tube and/or the second inner tube may be of the same or different materials. Noble metals can be understood to be metals, the redox pairs of which have a positive standard potential with respect to the normal hydrogen electrode. The noble metal may be platinum or the like. The noble metal alloy may be a platinum iridium alloy or the like, and in particular a PtIr10 or PtIr20 alloy.

In one embodiment, the sacrificial material of the first core is less noble than the material of the first inner tube. In one embodiment, the sacrificial material of the second core is less noble than the material of the first and/or second inner tube. Non-noble metals or base metals can be understood to be metals, the redox pairs of which have a negative standard potential with respect to the normal hydrogen electrode.

In one embodiment, the first core made of sacrificial material includes a base metal or a base metal alloy. In one embodiment, the second core made of sacrificial material includes a base metal or a base metal alloy. A base metal alloy can be understood to be an alloy of one or more base metals or non-noble metals. The sacrificial material of the first core and the sacrificial material of the second core may consist of or include the same or different materials. The base metal alloy may consist of or include copper, a nickel cobalt base alloy or the like. For better dimensional stability of the (smaller) opening to be produced, the sacrificial material of the second core may be harder than the sacrificial material of the first core. In one embodiment, the first core is made of copper. In one embodiment, the second core is made of a nickel cobalt base alloy. The nickel cobalt base alloy may be MP35N or MP35NLT. In one embodiment, the sacrificial material of the first core is selected from Cu, MP35N, Ni, Co, Ti, 316L, 301, 304, ceramic, or plastic. In one embodiment, the sacrificial material of the second core is selected from Cu, MP35N, Ni, Co, Ti, 316L, 301, 304, ceramic, or plastic.

In one embodiment, the sacrificial material of the first core and/or the sacrificial material of the second core include a base metal, in one embodiment copper. In one embodiment, the sacrificial material of the first core and/or the sacrificial material of the second core include a material selected from the list consisting of Cu, MP35N, Ni, Co, Ti, 316L, 301, 304, ceramic, and plastic.

The outer element, all inner elements and/or all sacrificial materials can in principle include different materials independently of one another. The material pairings can be chosen arbitrarily in such a way that the sacrificial material can be removed more easily than the surrounding inner element.

In one embodiment, the outer element includes a material selected from the list consisting of Pt, Ir, Ta, Pd, Ti, Au, W, Mo, MP35N, 316L, 301, 304 and Nb. In one embodiment, the outer tube includes a material selected from the list consisting of Pt, Ir, Ta, Pd, Ti, Au, W, Mo, MP35N, 316L, 301, 304 and Nb.

In one embodiment, the outer element includes a base metal, for example MP35N or a stainless steel alloy. Examples of stainless steel alloys are 316L, 301 and 304. In one embodiment, the outer tube includes a base metal, for example MP35N or a stainless steel alloy.

In one embodiment, the first inner element includes a material selected from the list consisting of Pt, Ir, Ta, Pd, Ti, Au, W, Mo, MP35N, 316L, 301, 304 and Nb.

In one embodiment, the first inner tube includes a material selected from the list consisting of Pt, Ir, Ta, Pd, Ti, Au, W, Mo, MP35N, 316L, 301, 304 and Nb.

In one embodiment, the first inner element includes a base metal, for example MP35N or a stainless steel alloy. In one embodiment, the first inner tube includes a base metal, for example MP35N or a stainless steel alloy.

In one embodiment, the second inner element includes a material selected from the list consisting of Pt, Ir, Ta, Pd, Ti, Au, W, Mo, MP35N, 316L, 301, 304 and Nb. In one embodiment, the second inner tube includes a material selected from the list consisting of Pt, Ir, Ta, Pd, Ti, Au, W, Mo, MP35N, 316L, 301, 304 and Nb. In one embodiment, the second inner element includes a base metal, for example MP35N or a stainless steel alloy. In one embodiment, the second inner tube includes a base metal, for example MP35N or a stainless steel alloy.

The outer tube and the first inner tube and optionally the second inner tube may each consist substantially of the same material or different materials. In the former case, however, it is provided that in any case the inner element which later forms a contacting opening additionally includes a material having a lower melting point. In one embodiment, the first inner tube and the second inner tube include a material each independently selected from the list consisting of Pt, Ir, Ta, Pd, Ti, Au, W, Mo, MP35N, 316L, 301, 304 and Nb.

In one embodiment, the first inner tube and the second inner tube consist of a material each independently selected from the list consisting of Pt, Ir, Ta, Pd, Ti, Au, W, Mo, MP35N, 316L, 301, 304 and Nb. In one embodiment, the first inner tube and the second inner tube consist of the same material, each independently selected from the list consisting of Pt, Ir, Ta, Pd, Ti, Au, W, Mo, MP35N, 316L, 301, 304 and Nb. In one embodiment, the first inner tube and the second inner tube consist of Pt or a Pt-containing alloy, for example PtIr10 or PtIr20.

In one embodiment, the outer element includes a noble metal, and the first inner element and/or the second inner element includes a non-noble metal. In one embodiment, the outer tube includes platinum, and the first inner tube and/or optionally the second inner tube includes titanium. In one embodiment, the outer tube includes platinum, and the first inner tube and/or optionally the second inner tube includes a non-noble metal.

In one embodiment, the manufacturing method includes cutting the composite tube into rings after removing the sacrificial materials. Cutting may be performed in a contact-free manner, for example by wire erosion. For cutting, the composite tube may be fixed with a clamping device and fastened, for example, to a strip.

In one embodiment, after removal of the sacrificial materials and either before or after cutting the composite tube into rings, the manufacturing method includes further processing, which in a longitudinal section through the ring electrode reduces the length of the second inner element in relation to the outer element and/or the first inner element, so that the second inner element does not extend in the longitudinal section along the entire length of the outer element and/or the first inner element. In other words, the second inner element or the contacting opening forms at least one step in the ring electrode. This can be done by mechanical machining and/or an erosion process.

After the removal of the sacrificial materials, a heat treatment and in particular a recrystallization annealing may be provided, for example in order to increase the ductility of the ring electrode.

The outer element and all inner elements may have any desired shapes in cross section and may, in particular, be circular, oval, elliptical, semicircular, but also square, rectangular, polygonal and the like. The outer element and all inner elements may have cross sections that differ from one another. In one embodiment, the outer element and all inner elements are circular in cross section.

In one embodiment, the outer tube and/or one or all of the inner tubes are profile tubes. A profile tube can be understood to be a tube which has a non-circular shape in cross section, such as, for example, a square, rectangular, semicircular or arc shape in cross section. In one embodiment, the first inner tube is a profile tube. In this case, the inner tube may be mostly circular in shape, but may have an arcuate bulge in at least one place which is designed to receive the second inner element. The profile tube may also have an arcuate bulge for a further inner element in a further place. The bulge of the profile tube may also be trapezoidal.

With the manufacturing method according to one embodiment, any numbers and arrangements of openings can be produced in a ring electrode. A through-opening may be formed in the ring electrode by removing the sacrificial material of the first core. A contacting opening for electrical and/or mechanical contacting may be formed by removing the sacrificial material of the second core. A further opening may be formed in the ring electrode by removing a sacrificial material of an optional third core. In one embodiment, the manufacturing method for this further includes the following steps:
Providing a third inner element including a third core made of a sacrificial material,
Forming the composite tube by arranging the third inner element within the outer element, the first, second, and third inner elements being arranged eccentrically to one another; and
Removing the sacrificial material of the third core.

The third inner element may have a third inner tube that includes a material having a lower melting point than the first material, and the third core made of sacrificial material. The sacrificial material of the third core may be removed by mordanting or etching as described above. The further opening of the ring electrode created by removing the third core may be arranged opposite the contacting opening created by removing the second core on the outer circumference of the first inner tube. The through-opening of the ring electrode created by removing the first core may be apple-shaped, so that the contacting opening and the further opening can each be arranged in the opposite bulges of the apple-shaped through-opening on the outer circumference of the contacting opening.

The material of the third inner tube may have a similar microstructure to the material of the outer tube, the material of the first inner tube, and/or the material of the second inner tube.

Further inner elements, each including a further inner tube and a further core made of sacrificial material, can be used in a corresponding manner to produce further contacting openings.

In one embodiment, an integral bond between the outer element and the first inner element and optionally the second inner tube is formed by drawing and/or heating the composite tube.

In one embodiment, the outer element and the first inner element, and optionally the second inner tube, are joined by the heating so as to form a material composite having a substantially uniform microstructure.

In one embodiment, the method further includes drawing the composite tube again in a longitudinal direction of the composite tube following the heating of the composite tube described above. Smaller sized ring electrodes can be produced, as described herein, by such anew drawing after connecting the outer element to the inner element.

In one embodiment, the outer tube, the first inner tube, and optionally the second inner tube include a noble metal.

One embodiment further relates to a ring electrode which can be produced according to a method described herein.

It is further proposed to provide a ring electrode including an outer element, a first inner element, and a second inner element. The outer element includes an outer tube that includes a first material. The first inner element and the second inner element are arranged within the outer element. The first inner element and the second inner element are arranged eccentrically to one another to form a composite tube. The outer element, the first inner element, and the second inner element have been drawn together in a longitudinal direction of the composite tube. The first inner element has a first inner tube surrounding a first cavity from which a sacrificial material has been removed. The second inner element surrounds a second cavity from which a sacrificial material has been removed and which forms a contacting opening in the ring electrode. The second inner element includes a second material having a lower melting point than the first material. The ring electrode is in one embodiment designed to produce a mechanical and electrically conductive connection to a conductor element by using heating the second material. The heating of the second material is in one embodiment a heating to a temperature below the first material.

### Ring Electrode Precursor

One embodiment further relates to a precursor for producing a ring electrode which includes an outer element and an inner element, which is arranged eccentrically within the outer element and is directly connected thereto, wherein the outer element includes an outer tube made of a first material, and the inner element includes an inner tube made of a second material, the second material having a lower melting point than the first material.

The outer element in one embodiment includes a tubular element, also referred to herein as outer tube. The outer element includes a through-opening, which is in one embodiment configured to receive a cable, the cable in one embodiment including a plurality of electrical conductors, referred to herein as conductor elements. The individual conductor elements may include an electrical insulation on their outer side. The ring electrode includes an inner element which is mechanically and electrically conductively connected to the outer element. The inner element is in one embodiment tubular and may include a tubular element, also referred to herein as inner tube. The inner element includes a second material having a lower melting point than the first material. In one embodiment, the inner tube consists of the second material.

In one embodiment, the inner tube surrounds a core made of a sacrificial material, and the outer element surrounds a further core made of a sacrificial material.

The precursor can furthermore include a further inner element which includes a further inner tube and optionally a further core within the further inner tube.

For further details of the precursor, reference is made to the description of the manufacturing method herein.

In one embodiment, a material of the outer element and a material of the first inner element have a microstructure similar to each other.

For example, the material of the outer tube and the material of the first inner tube may have a microstructure similar to each other. This means, for example, that in each case the crystal grains of a metal have a similar size and/or shape in both materials.

In one embodiment, the A:B ratio is from 0.8 to 1.2; in one embodiment from 0.9 to 1.1; from 0.95 to 1.05, or from 0.99 to 1.01, wherein A is the average crystal grain size of the outer tube and B is the average crystal grain size of the first inner tube. The grain size can be determined with the test methods described hereinafter.

In some embodiments, the C:D ratio is from 0.8 to 1.2; in one embodiment from 0.9 to 1.1; from 0.95 to 1.05, or from 0.99 to 1.01, wherein C is the hardness of the material of the outer tube and D is the hardness of the material of the inner tube. Vickers hardness can be determined by the test methods described hereinbelow.

In a further embodiment, the precursor includes an outer element, a first inner element, and a second inner element, the outer element including an outer tube, wherein the first inner element includes a first inner tube having an outer side, the first inner element and the second inner element being arranged within the outer element, and the first inner element and the second inner element being arranged eccentrically to one another to form a composite tube, wherein the outer element includes a first material, and the inner element includes a second material, the second material having a lower melting point than the first material.

### Test Methods

In the absence of specifically mentioned measurement conditions, all measurements are taken under standard conditions, i.e. at a temperature of 298.15 K and an absolute pressure of 100 kPa.

### Hardness

Hardness is the mechanical resistance that a material provides to mechanical penetration by another body. Hardness can be measured by microindentation. Here, a diamond test body according to Vickers is pressed into the layer and the force-displacement curve is recorded during the measurement. The curve can then be used to calculate the mechanical properties of the specimen, including hardness. Hardness can be determined, for example, with the Anton Paar MHT-10 Microhardness Tester. It should be noted that the impression depth should not be more than 10% of the layer thickness, since otherwise properties of the substrate may distort the measurements. Hardness according to Vickers can be determined according to the standard DIN EN ISO 6507-4:2018.

### Grain Size

To measure the crystal grain size, a first cross section of the sample to be examined is prepared using metallographic methods. The grain size of the first cross-section is then measured using a light microscope (Leica DM4000). The light microscope is used to generate a two-dimensional first image of the grain structure of the sample. The grain size of 100 grains is measured. If the first image includes less than 100 grains, another image is generated by creating a further cross-section of the sample. The average grain size is calculated from the arithmetic average of the 100 grain sizes. The grain size is defined as the maximum linear distance that can be measured between 2 points on the grain boundary. For example, if the grain has an elongate shape, the grain size should be measured in the longest direction.

Furthermore, the grain boundary may have a certain width. The width of the grain boundaries is not included in the calculation of the grain size.

### EXAMPLES

One embodiment is further illustrated below using examples, which, however, are not to be understood as limiting. It will be apparent to those skilled in the art that other equivalent means may be similarly used in place of the features described herein.

The figures illustrate, by way of example, various intermediate and end products of the method according to one embodiment and ring electrodes 10 according to one embodiment. The ring electrode 10 can be used as an active implantable medical device, for example in a cardiac pacemaker or for neurostimulation. It can be used for signal sensing and for stimulation.

A manufacturing method for the ring electrode 10 includes the following steps here (not necessarily in this order):
In a step S1, Providing an outer element 11 that includes an outer tube 12 which includes a first material.
In a step S2, Providing a first inner element 13 that includes a first inner tube 14 having a first core 15 made of a sacrificial material.
In a step S3, Providing a second inner element 16 that includes a second material having a lower melting point than the first material, the inner element further including a second core 17 made of a sacrificial material.
In a step S4, Forming a composite tube by arranging the first inner element 13 and the second inner element 16 within the outer element 11, the first inner element 13 and the second inner element 16 being arranged eccentrically to each other.
In a step S5, Drawing the composite tube in a longitudinal direction of the composite tube.
In a step S6, Separating a composite tube disc from the composite tube,
In a step S7, Removing the sacrificial material of the first core 15 to obtain a ring electrode 10.
In a step S8, Removing the sacrificial material of the second core 17 to obtain a contacting opening in the ring electrode 10.

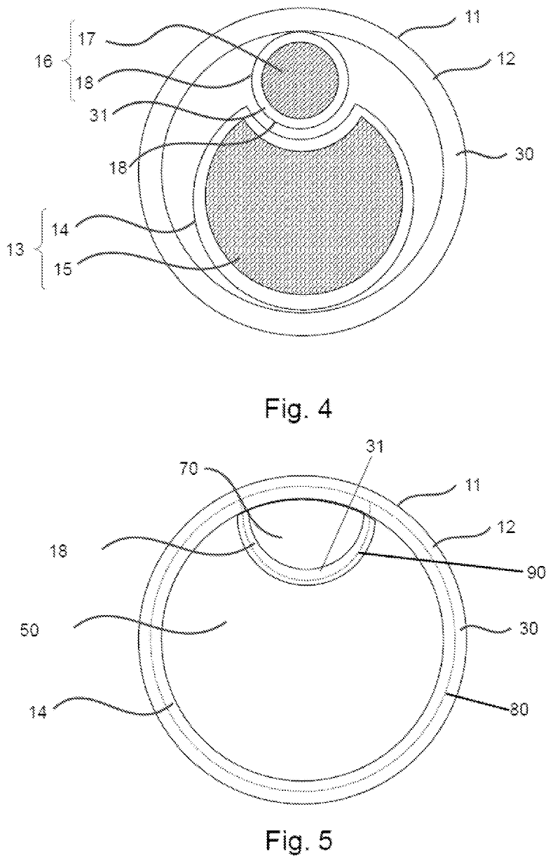

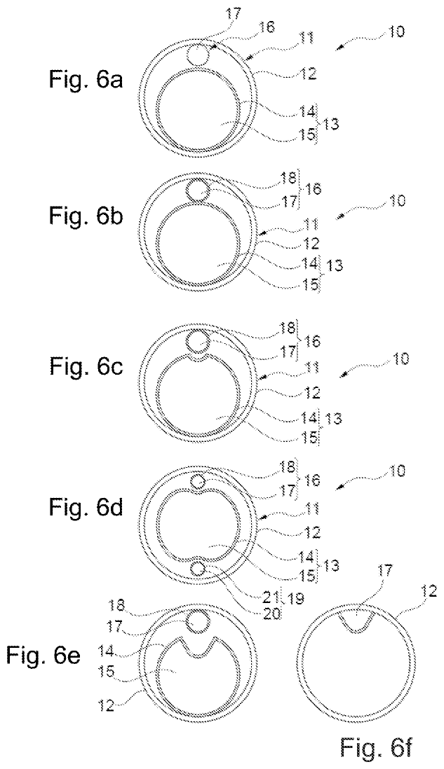

Exemplary intermediate products of the method described above are described in more detail below in FIGS. 4 and 6.

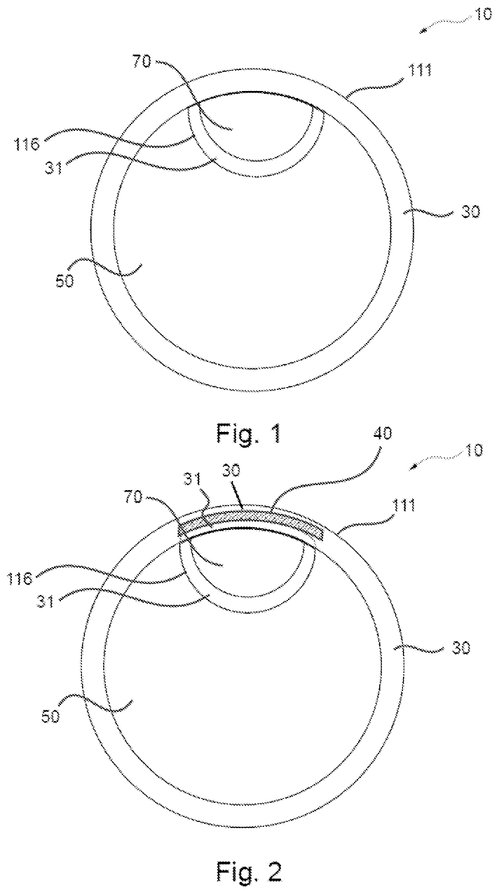

FIG. 1 illustrates a cross-sectional drawing of a ring electrode 10 according to one embodiment. The ring electrode 10 includes a tubular exterior wall 111 consisting of a first material 30, here PtIr10. A contact element 116 which includes a second material 31, here gold, is arranged within the exterior wall 111. In particular, the inner side of the contact element 116 consists entirely of the second material 31. The inner side of the exterior wall 111 and the outer side of the contact element 116 jointly define a through-opening 50 configured to receive a cable. The inner side of the contact element 116 defines a contacting opening 70 which is designed for receiving and electrically conductively connecting to a conductor element (not illustrated in FIG. 1). By heating the second material to a temperature below the melting point of the first material, in one embodiment to a temperature above the melting point of the second material, a conductor element can be connected to the ring electrode 10 within the contacting opening 70.

FIG. 2 illustrates a cross-sectional drawing of a ring electrode 10 according to one embodiment, which in addition to the elements illustrated in FIG. 1 includes a diffusion barrier 40 consisting of nickel in this example. The exterior wall 111 includes the first material and the second material. The first material is arranged on the outer side of the exterior wall 111. The second material is arranged on the inner side of the exterior wall 111. The diffusion barrier is positioned within the exterior wall 111 so as to separate the first material within the exterior wall and the second material within the exterior wall from one another. The diffusion barrier 40 is arranged and configured to completely or partially prevent diffusion of the second material into the first material within the exterior wall 111, particularly when the second material is heated to connect to a conductor element.

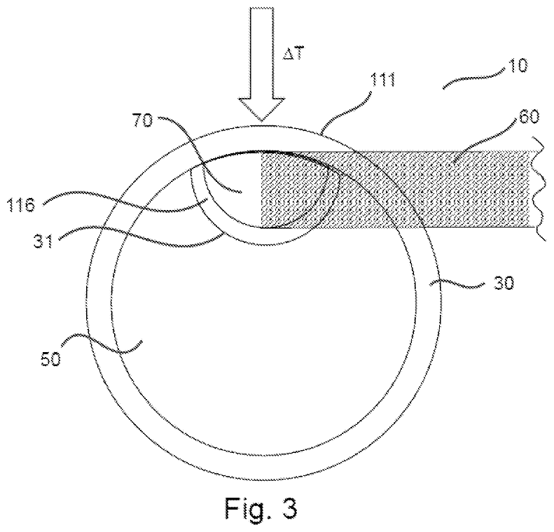

FIG. 3 illustrates a cross-sectional drawing of a ring electrode 10 according to one embodiment, which is connected to a conductor element 60 by the action of heat from outside. The conductor element 60 is inserted into the contacting opening 70 of the contact element 116. The second material 31 is heated above the melting point of the second material by the action of heat on the exterior wall 111, here by using a laser welding method, so as to bond integrally to the conductor element 60. The temperature in this process is below the melting point of the first material 30 so that the structural integrity of the exterior wall 111 is not impaired.

FIG. 4 illustrates a cross-sectional drawing of a precursor for producing a ring electrode. The precursor includes an outer element 11 including an outer tube 12. The outer tube consists of a first material, here PtIr10. A first inner element 13 and a second inner element 16 are each arranged eccentrically within the outer element 11. The first inner element contains a first inner tube 14 surrounding a first core 15. The second inner element 16 includes a second inner tube 18 surrounding a second core 17. Here, the first inner tube and the second inner tube consist of PtIr10. Here, the first core and the second core consist of copper. The first inner element 13 includes an indentation in which the second inner element 16 is partially arranged.

FIG. 5 illustrates a cross-sectional drawing of a ring electrode which can be produced from a precursor illustrated in FIG. 4. The outer element, the first inner element and the second inner element from FIG. 4 have been formed into a one-piece material composite by using drawing. The outer tube 12 is directly connected to the first inner tube 14, these two elements forming a first boundary surface 80 in the exterior wall of the electrode. The first inner tube 14 is connected directly to the second inner tube 18 in the region of the contacting opening 70, wherein these two elements form a second boundary surface 90 in the region of the contacting opening 70 which extends in the direction of the center or main axis of the ring electrode. The first boundary surface 80 and the second boundary surface 90 are each arranged between the first material and the second material.

FIGS. 6a to 6e illustrate top views of several embodiments of a precursor of the ring electrode 10 after forming the composite tube, but before drawing the composite tube. The precursor of the ring electrode 10 includes an outer element 11, a first inner element 13 and a second inner element 16.

In the embodiment illustrated in FIG. 6a in particular, the outer element 11 is circular and includes a circular outer tube 12. The first inner element 13 and the second inner element 16 are also circular and lie within the outer element 11 and its outer tube 12. The first inner element 13 and the second inner element 16 are arranged eccentrically to one another, i.e. the center points of the two inner elements do not lie on top of each other. The diameter of the first inner element 13 is significantly larger than the diameter of the second inner element 16.

The first inner element 13 has a circular first inner tube 14 surrounding a likewise circular first cavity that includes a first sacrificial material. The second inner element 16 surrounds a circular second cavity that includes a second sacrificial material. Here, the outer tube 12 and the first inner tube 13 consist of the alloy PtIr10. Here, the first core 15 consists of steel and is completely coated with copper on the outer side. Here, the second core 17 consists of 316L. By removing the sacrificial material of the first core 15, a through-opening may be produced in the ring electrode 10 in the subsequent manufacturing step S7. Step S7 may be mordanting with hydrochloric acid in an ultrasonic bath at 80° C. By removing the sacrificial material of the second core 17, a contacting opening for electrical and/or mechanical contacting may be produced in the subsequent manufacturing step S8. Step S8 may be mordanting with FeCl₃ in for 15 minutes in an ultrasonic bath at 60° C. The contacting opening may serve as an electrical connecting element and/or as a mechanical fastening element for a conductor element to form an electrode system of the ring electrode 10 and the conductor element.

In the embodiment illustrated in FIG. 6b, the second inner element 16 includes a second inner tube 18 that includes the second core 17. When drawing the composite tube, the second inner tube 18 can flow into free spaces between the outer tube 12 and the first inner tube 13. Here, the second inner tube 18 consists of copper.

In the embodiments illustrated in FIGS. 6c to 6e, the first inner tube 13 is a profile tube. The inner tube 13 is mostly circular in shape, but has an arcuate (FIG. 2c) or trapezoidal (FIG. 2e) bulge in one place in the embodiments illustrated in FIGS. 2c and 2e in order to receive the second inner element 16.

In the embodiment according to FIG. 6c, the core 17 of the second inner tube 18 is coated with copper, and the second inner tube 18 consists of copper. In the embodiment illustrated in FIG. 6d, the profile tube of the first inner tube 13 has an arcuate bulge for a further, third inner element 19 in a further place opposite the second inner element 16. The third inner element 19 lies within the outer element 11, and the first, second, and third inner elements are arranged eccentrically to one another. The third inner element 19 includes a third inner tube 21 and a third core 20 made of a sacrificial material, the removal of which may produce a further opening in the ring electrode 10. In the embodiment illustrated in FIG. 2d, the removal of the first core 15 creates an apple-shaped through-opening of the ring electrode 10 in which the contacting opening and the further opening are each arranged in the opposite bulges of the apple-shaped through-opening 3. A coating of copper is arranged on the inner side of the second inner tube 18. A coating of copper is arranged on the inner side of the third inner tube 21.

In the embodiment illustrated in FIG. 6e, the first inner tube 14 consists of copper, and the second inner tube 18 consists of copper.

In FIG. 6f illustrates a top view of a precursor of the ring electrode 10 after step S5, the drawing of the composite tube in a longitudinal direction of the composite tube. The outer element and the inner elements are connected to form a one-piece material composite. On its inner side, the contacting opening includes a material with a lower melting point than a material of the exterior wall, in particular of the outer tube 12.

Although specific embodiments have been illustrated and described herein, it will be appreciated by those of ordinary skill in the art that a variety of alternate and/or equivalent implementations may be substituted for the specific embodiments illustrated and described without departing from the scope of the present embodiment. This application is intended to cover any adaptations or variations of the specific embodiments discussed herein. Therefore, it is intended that this embodiment be limited only by the claims and the equivalents thereof.

1. A ring electrode for electrical stimulation or sensing on the human body comprising:
an exterior wall; and
a contact element directly connected to the exterior wall, the contact element being arranged eccentrically within the exterior wall;
wherein the exterior wall comprises a first material and the contact element comprises a second material;
wherein the second material has a lower melting point than the first material;
wherein the exterior wall comprises a through-opening; and
wherein the contact element comprises a contacting opening for connection to a conductor element.

2. The ring electrode according to claim 1, wherein the second material is selected from the group consisting of Pt, Cu, Pd, Ti, Fe, Au, Mo, Ni, MP35N, 316L, 301, 304, and an active solder.

3. The ring electrode according to claim 1, wherein the first material is selected from the group consisting of Pt, Ir, Ta, Pd, Ti, Fe, Au, Mo, Nb, W, Ni, Ti, MP35N, 316L, 301 and 304.

4. The ring electrode according to claim 1, further comprising a diffusion barrier between the first material and the second material.

5. The ring electrode according to claim 1, wherein the absolute melting point of the first material is at least 1.1 times the absolute melting point of the second material.

6. The ring electrode according to claim 1, wherein the absolute melting point of the first material is at least 2 times the absolute melting point of the second material.

7. An electrode system comprising a ring electrode according to claim 1 and a conductor element, wherein the conductor element is arranged within the contacting opening and is integrally bonded to the contact element and is alloyed therewith.

8. A method for connecting a ring electrode according to claim 1 to a conductor element, comprising:
(i) bringing the ring electrode into contact with the conductor element, the conductor element being arranged at least partially within the contacting opening,
(ii) heating the contact element and thereby forming an integral bond between the second material and the conductor element, wherein the formation of an integral bond preferably comprises the formation of an alloy.

9. The method according to claim 8, wherein heating of the contact element is effected by heating the outer side of the exterior wall.

10. The method according to claim 8, wherein heating of the contact element is effected by induction heating or local heat input by a laser beam or resistance welding.

11. The method according to claim 8, further comprising compressing the conductor element within the contacting opening to produce a frictional connection between the conductor element and the contact element.

12. An electrode system produced by the method according to claim 8.

13. A manufacturing method for a ring electrode, comprising:
(a) providing an outer element comprising an outer tube made of a first material,
(b) providing a first inner element comprising a first inner tube and a first core made of a sacrificial material,
(c) providing a second inner element comprising a second inner tube made of a second material and a second core made of a sacrificial material, said second material having a lower melting point than the first material,
(d) forming a composite tube by arranging the first inner element and the second inner element within the outer element, the first inner element and the second inner element being arranged eccentrically to each other,
(e) drawing the composite tube in a longitudinal direction of the composite tube,
(f) separating a composite tube disc from the composite tube,
(g) removing the sacrificial material of the first core, and
(h) removing the sacrificial material of the second core to obtain a contacting opening in the ring electrode.

14. A precursor for a ring electrode, comprising an outer element and an inner element which is arranged eccentrically within the outer element and is directly connected thereto, said outer element comprising an outer tube made of a first material,
and said inner element comprising an inner tube made of a second material, said second material having a lower melting point than the first material.

15. The precursor for a ring electrode according to claim 14, wherein the inner tube surrounds a core made of a sacrificial material and the outer element surrounds a further core made of a sacrificial material.

16. The precursor for a ring electrode according to claim 14, further comprising a further inner element comprising a further inner tube and optionally a further core within the further inner tube.
